(19) United States
(12) Plant Patent Application Publication
(10) Pub. No.: US 2009/0113588 P1
(43) Pub. Date: Apr. 30, 2009

(54) Athyrium plant named 'Ocean's Fury'

(50) ;

(76) Inventor: Thurman Maness, Pittsboro, NC (US)

Correspondence Address: KLARQUIST SPARKMAN, LLP, 121 SW SALMON STREET, SUITE 1600, PORTLAND, OR, 97204, US

(21) Appl. No.: 11/980,128

(22) Filed: Oct. 29, 2007

(51) Int. Cl. A01H 5/00

(52) U.S. Cl. PLT/373

(57) ABSTRACT

A new and distinct cultivar of Athyrium niponicum var. pictum plant characterized in having a strongly upright habit, and leaves of a pewter tone, silver over grey green, with crested tips, and a reddish rachis. This coloring persists from new growth to mature leaves.

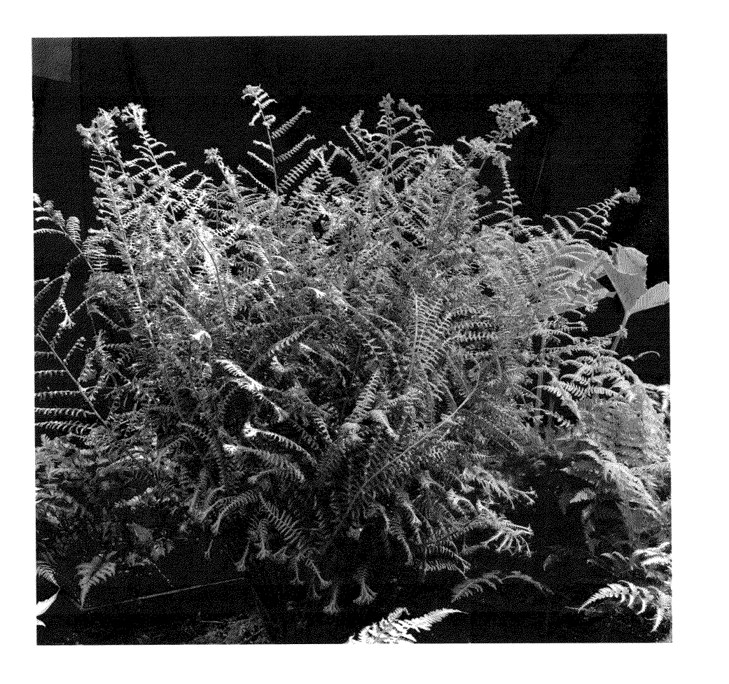

### BOTANICAL CLASSIFICATION

Athyrium spp.

### VARIETY DENOMINATION

‘Ocean's Fury’

### PARENTAGE

Athyrium niponicum var. pictum and Athyrium filix-femina ‘Congestum Cristatum’ (unpatented)

### BACKGROUND OF THE INVENTION

The present invention relates to a new and distinct Athyrium, and given the cultivar name of ‘Ocean's Fury’. Athyrium is in the family Woodsiaceae. This new cultivar originated from a cross between Athyrium niponicum var. pictum and Athyrium filix-femina ‘Congestum Cristatum’.

The new variety has been reproduced only by asexual propagation (tissue culture). Each of the clones exhibits identical characteristics to the original selection.

### SUMMARY OF THE INVENTION This plant is unique in having:

1. A strongly upright habit, and leaves of a pewter tone; and
2. Silver over grey green, with crested tips, and a reddish rachis. This coloring persists from new growth to mature leaves.

Asexual propagation by tissue culture as done in Canby, Oreg., shows that the foregoing characteristics and distinctions come true to type and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

The photograph shows a three-year-old Athyrium ‘Ocean's Fury’ growing in the ground in a shaded site in Canby, Oreg.

### DETAILED PLANT DESCRIPTION

The following is a detailed description of the new Athyrium based on observations of four-year-old specimens grown in the ground in a shade structure in Canby, Oreg. The color descriptions are all based on The Royal Horticultural Society Colour Chart.
Plant:
   Form.—terrestrial, clumping.
   Hardiness.—USDA Zones 5 to 8.
   Size.—80 cm wide and 85 cm tall.
Foliage:
   Type.—compound.
   Shape.—lanceolate to elliptic overall, twice pinnate, pinnae lanceolate with a crested tip, pinnules lanceolate to linear with a crested tip, pinnules pinnatifid.
   Size.—grows to 85 cm long and 27 cm wide, pinnae grow to 19 cm long and 4 cm wide, pinnules grow to 2 cm long and 3 mm wide.
   Margins.—pinnules pinaatifid.
   Apex.—crested at apex of pinnae with main vein splitting into two twice and branching out and pinnules to a lesser degree.
   Surface texture.—glabrous on both sides.
   Leaf color.—Topside: pinnule centers Yellow Green 148C, sides Yellow Green 147B, and tips Yellow Green 147A. Bottom side: crests Yellow Green 147A, sori Brown 200D.
   Rachis color.—Greyed Red 182A on top side and Yellow Green 152B on back side.
   Petiole description.—grows to 20 cm long and 5 mm wide, glabrous with chaffy scales (Greyed Orange 175A), heaviest at the base, Brown 200C on side closest to plant center and Yellow Green 152B on back side.
Fruiting body:
   Type.—sporangium.
   Fertility.—very low.
   Sori.—in clusters on ultimate pinnules, indusium on side closest to the margin Size — 1 to 2 mm at the widest Immature — Brown 200D Sporulating — Brown 200D.
Diseases and pests: Some Athyrium are prone to rust. No diseases have been observed on plants grown commercially in Canby, Oreg. Athyrium generally have no pest problems.
Comparisons: The instant plant resulted from a cross between Athyrium niponicum var. pictum and Athyrium Filix-femina ‘Congestum Cristatum’ (unpatented). Compared to Athyrium niponicum var. pictum, Athyrium ‘Ocean's Fury’ has very different foliage shape. It does show the coloration of the Japanese Painted Fern with the red in the stipe and the grey green tones in the leaves. Compared to its other parent Athyrium filix-femina ‘Congestum Cristatum’, the new variety has different leaf shape and coloring but does show the cresting.

I claim:

1. A new and distinct Athyrium plant substantially as shown and described.
